SEQUENCE LISTING (2 sequences)

SEQ ID NO: 1 - DNA, 41 bp, Artificial Sequence
  primer A′
tttttttttttttttgtcggagacacgcagggatgagatgg

SEQ ID NO: 2 - DNA, 26 bp, Artificial Sequence
  primer A
ccatctcatccctgcgtgtctccgac

(19) United States
(12) Patent Application Publication
(10) Pub. No.: US 2014/0350235 A1
(43) Pub. Date: Nov. 27, 2014

(54) IN SITU OLIGONUCLEOTIDE SYNTHESIS ON A PARAMAGNETIC SUPPORT

(71) Applicants: Michael S. Akhras, Stockholm (SE); Michael A. Jensen, Los Gatos, CA (US)

(72) Inventors: Michael S. Akhras, Stockholm (SE); Michael A. Jensen, Los Gatos, CA (US)

(73) Assignee: The Board of Trustees of the Leland Stanford Junior University, Palo Alto, CA (US)

(21) Appl. No.: 14/280,609

(22) Filed: May 18, 2014

Related U.S. Application Data
(60) Provisional application No. 61/826,963, filed on May 23, 2013.

(51) Int. Cl. C07H 1/02; H01F 13/00; B01J 19/08

(52) U.S. Cl. CPC C07H 1/02; B01J 19/087; H01F 13/003; H01F 13/006; B01J 2219/085
USPC 536/25.34; 422/186.01; 335/284

(57) ABSTRACT

A novel method for attaching oligonucleotides to a paramagnetic solid support is disclosed. Conventional methods of attachment require that oligonucleotides be pre-synthesized with specific end modifications, which is laborious and expensive. Instead, we attached oligonucleotides to paramagnetic beads by direct synthesis of the oligonucleotides on the surface of the beads. An external magnet was used to hold the paramagnetic beads in place during solid-phase synthesis. A magnetic force was applied directly to the beads to prevent their loss, in particular, during reagent purge-to-waste steps that involved high-pressure drain or vacuum. This method can be adapted for use in any laboratory working with conventional synthesis automation, and can be employed, for example, with single columns and multi-well titer plates.

### CROSS-REFERENCE TO RELATED APPLICATION

This application claims benefit under 35 U.S.C. §119(e) of provisional application Ser. No. 61/826,963, filed May 23, 2013, which is hereby incorporated by reference in its entirety.

### STATEMENT REGARDING FEDERALLY SPONSORED RESEARCH OR DEVELOPMENT

This invention was made with Government support under contract HG000205 awarded by the National Institutes of Health. The Government has certain rights in this invention.

### TECHNICAL FIELD

The present invention pertains generally to the chemical synthesis of oligonucleotides. In particular, the invention relates to a method of synthesizing oligonucleotides directly on a solid paramagnetic support that is immobilized by a magnet.

### BACKGROUND

Micro- and nanometer-sized beads have been extensively used for a vast variety of molecular diagnostic assays (Haukanes et al. (1993) Biotechnology 11:60-63; Holmberg et al. (2005) Electrophoresis 26:501-510). Such assays follow the same general scheme: Each individual bead carries a surface modification that is capable of binding a particular type of ligand (chemical group or compound). The beads can then either be used for non-target specific purposes (e.g. carboxyl groups—nucleic acids and streptavidin-biotin) or target-specific purposes by surface coupling of specific biomarker biomolecules (e.g., nucleic acids and antibody/antigens)(Nakajima and Ikada (1995) Bioconjug. Chem. 6:123-130; Gilles (1990) Anal. Biochem. 184:244-248; Sehgal and Vijay (1994) Anal. Biochem. 218:87-91; Szajáni et al. (1991) Appl. Biochem. Biotechnol. 30:225-231). For nucleic acid ligands, the oligonucleotides must be pre-synthesized with specific end modification to fit the bead-attachment chemistry used. The biomarker molecule ligand will have affinity for a corresponding target molecule enabling selection of the target for downstream processes (Nolan (2001) Trends Biotechnol. 20:9-12; Dunbar (2006) Clinica Chimica Acta 363:71-82).

Paramagnetic beads, compared to other bead-types (e.g. acrylamide, sepharose, silica, and polystyrene), provide a major advantage in sample preparation methodology because of the inherent ease of separating paramagnetic particles in solution with an external magnet (Holmberg et al., supra). Paramagnetic beads have been used for various types of sample preparation and detection platforms including: i) pyrosequencing (Holmberg et al., supra; Ronaghi (2001) Genome research 11:3-11; qiagen.com), ii) Luminex(8)(luminex.com), iii) Applied Biocode (appliedbiocode.com), iv) SOLID (Shendure (2005) Science 309:1728-1732; lifetechnologies.com), and v) MagArray platform (Xu et al. (2008) Biosensors and Bioelectronics 24:99-103, magarray.com).

Furthermore applications using paramagnetic bead nucleic acid capture and isolation are becoming more sophisticated with smaller bead diameters, including nanoparticles being used (Xu et al., supra). Traditional solid-phase chemical oligonucleotide synthesis utilizes a filter-based platform that cannot retain submicron beads. The commercial synthesis substrates, such as controlled-pore glass (CPG) particles or polystyrene beads, have a median diameter of about 100 μm, and the filters used to retain these substrates have a median porosity of 40 μm, which allows only passage of organic solvents during wash and drain steps of the synthesis cycle. Thus, these filters are obviously too porous for the popular MyOne beads, which are only 1 μm in diameter. Conversely, filters with less than 2 μm porosity restrict reagent flow-through, and cannot be used in synthesis. As a result, past investigations of chemical syntheses on paramagnetic beads have had only limited, small-scale success that has proved impossible to adapt to higher throughput commercial synthesis automation (Albretsen et al. (1990) Anal. Biochem. 189:40-50).

Thus, there remains a need for improved methods of chemical synthesis of oligonucleotides on paramagnetic beads.

### SUMMARY

The present invention relates to a novel method for attaching oligonucleotides to a paramagnetic solid support by direct synthesis of the oligonucleotides on the surface of the paramagnetic solid support. An external magnet, which attracts the paramagnetic solid support, is used to hold the paramagnetic solid support in place during synthesis. This method can be adapted for use in any laboratory working with conventional synthesis automation, and can be employed, for example, with synthesis columns and multi-well titer plates.

In one aspect, the invention includes a method of synthesizing an oligonucleotide, the method comprising: a) providing a paramagnetic solid support comprising a substrate coating the surface of the solid support, wherein the substrate comprises one or more functional groups capable of covalent attachment to a linker; b) providing a magnet that attracts the paramagnetic solid support, whereby the paramagnetic solid support is immobilized on the surface of the magnet or a surface in contact with the magnetic field of the magnet; c) covalently attaching one or more phosphoramidite linkers to functional groups on the substrate to produce a derivatized support; d) reacting the derivatized support with a nucleoside phosphoramidite corresponding to the first nucleotide of the desired oligonucleotide sequence such that the nucleoside phosphoramidite attaches covalently to a phosphoramidite linker; and e) adding nucleoside phosphoramidites stepwise to the growing nucleotide chain until an oligonucleotide having the desired sequence is produced. Oligonucleotide synthesis may be performed in either the 5′ to 3′ direction or the 3′ to 5′ direction by suitable choice of nucleoside phosphoramidite reagents (e.g., 3′-[(2-cyanoethyl)-(N,N-diisopropyl)]-phosphoramidites for synthesis in the 3′ to 5′ direction or 5′-[(2-cyanoethyl)-(N,N-diisopropyl)]-phosphoramidites (Glen Research) for synthesis in the 5′ to 3′ direction). The method may further comprise cleaving the oligonucleotide from the support.

The paramagnetic solid support may comprise, for example, paramagnetic beads or non-spherical paramagnetic particles of any shape, including paramagnetic microparticles or nanoparticles. In certain embodiments, the paramagnetic solid support comprises a superparamagnetic material. The paramagnetic solid support may further comprise an inert polymeric coating on the surface of the support, including, but not limited to Teflon, perfluoroalkoxy, fluorinated ethylene propylene copolymer or polystyrene to protect the support from corrosion during oligonucleotide synthesis and to prevent exposure of the incipient oligonucleotide to metal ions from the interior of the paramagnetic solid support.

In certain embodiments, the substrate functional group used for attachment of the linker is a hydroxyl group or an amino group. For example, a hydroxylated substrate, such as hydroxylated polystyrene may be used for attachment of the linker. In certain embodiments, the phosphoramidite linker is between 30 and 60 atoms in length. Exemplary phosphoramidite linkers that can be used in the practice of the invention include 3-(4,4′-dimethoxytrityloxy)propyl-1-[(2-cyanoethyl)-(N,N-diisopropyl)]-phosphoramidite (C3), 9-O-dimethoxytrityl-triethylene glycol, 1-[(2-cyanoethyl)-(N,N-diisopropyl)]-phosphoramiditepropanediol (C9), and 18-O-dimethoxytritylhexaethyleneglycol, 1-[(2-cyanoethyl)-(N,N-diisopropyl)]-phosphoramidite (C18).

The magnet may be added to a container (e.g., column, microcentrifuge tube, test tube, or titer plate well) where oligonucleotide synthesis is carried out to immobilize the paramagnetic support during synthesis. In certain embodiments, the magnet is a magnetic sphere or magnetic disc 1 mm to 6 mm in diameter. Alternatively, the magnet may surround a container or be placed in proximity to a container, such that the paramagnetic solid support is immobilized on a surface of the container. In one embodiment, the magnet is a magnetic disc having an inner hole of a diameter designed to fit around a container in which oligonucleotide synthesis is performed. For example, the magnetic disc can be designed to fit around a column in which oligonucleotide synthesis is performed. In another embodiment, the magnet is a magnetic sleeve comprising a plurality of holes of a diameter designed to fit around a plurality of containers in which oligonucleotide synthesis is performed. For example, the magnetic sleeve can be designed to fit around wells of a multi-well plate (e.g., 96 well plate, 384 well plate, or 1,536 well plate) or around microcentrifuge tubes or test tubes.

In certain embodiments, the method further comprises temporarily demagnetizing the magnet to release the paramagnetic solid support from the magnet. For example, the magnet can be demagnetized by switching the magnetic poles of the magnet by moving the magnet back and forth near an external magnet or exposing the magnet to alternating current. In one embodiment, the magnet is passed through a conducting coil carrying an alternating current such that the magnet becomes demagnetized. Alternatively, the magnet can be demagnetized by heating the magnet to a temperature at or above the Curie point of the magnet. If the paramagnetic solid support is immobilized during oligonucleotide synthesis by indirect contact with the magnet, that is, by immobilization on a surface in contact with the magnetic field of the magnet (e.g., surface of a column, tube, or titer well surrounded by the magnet or next to or in contact with the magnet), the paramagnetic solid support can be released from the surface simply by removing the magnet.

In another aspect, the invention includes an automated system capable of synthesizing one or more oligonucleotides according to the methods described herein. Oligonucleotide synthesis can be automated by use of a commercial nucleic acid synthesizer or automated laboratory workstation. For example, commercially available automated nucleic acid synthesizers (e.g., Applied Biosystems synthesizers AB394 and AB3900) and automated laboratory workstations (e.g., Beckman Coulter Biomek FX) can be adapted to prepare oligonucleotides of interest with modifications to or addition of software instructing the instrument what reagents to add in a specified order and providing for control of a magnet. In one embodiment, a commercial automated system is modified to include a conducting coil carrying an alternating current capable of demagnetizing a magnet. In another embodiment, the commercial automated system is modified to include a magnetic bracket. The automated system may include one or more removable magnets capable of attaching to one or more containers in which oligonucleotide synthesis is performed. In certain embodiments, the automated system comprises at least one magnetic disc comprising an inner hole of a diameter designed to fit around a container in which oligonucleotide synthesis is performed. In another embodiment, the automated system comprises at least one magnetic sleeve comprising a plurality of holes of a diameter designed to fit around a plurality of containers in which oligonucleotide synthesis is performed. For example, the magnetic sleeve may be designed to fit around wells of a multi-well plate (e.g., 96 well plate, 384 well plate, or 1,536 well plate), microcentrifuge tubes, or test tubes.

In another aspect, the invention includes a device for reversibly demagnetizing and remagnetizing a magnet for controlling release of a paramagnetic solid support from the magnet or reattachment of the paramagnetic solid support to the magnet, respectively. The device comprises: a) a housing; b) a conducting coil positioned inside the housing, wherein said conducting coil is capable of generating a magnetic field, whereby the magnet when positioned within the magnetic field is demagnetized when alternating current (AC) flows through the conducting coil and remagnetized when direct current (DC) flows through the conducting coil; c) an AC power supply; d) a rectifier that converts AC to direct current (DC); and e) a circuit connected to the AC power supply and the conducting coil and having a switch that controls whether or not AC from the AC power supply passes through the rectifier before flowing through the conducting coil, wherein the position of the switch determines whether AC or DC is supplied to the conducting coil. The device may further comprise a platform positioned such that the platform is in contact with the magnetic field generated by current flowing through the conducting coil, wherein the platform can be used for placement of a reaction container for performing synthesis on a paramagnetic solid support, as described herein. In certain embodiments, the device is incorporated into an automated system, such as a commercial nucleic acid synthesizer or automated laboratory workstation.

In another aspect, the invention includes a kit comprising one or more magnets and reagents (e.g., nucleoside phosphoramidites, phosphoramidite linkers, and paramagnetic beads) or devices for performing oligonucleotide synthesis according to a method described herein. The kit may include magnets in various forms, such as, but not limited to magnetic spheres, magnetic discs, magnetic brackets, and magnetic sleeves. In one embodiment, the kit comprises at least one magnetic disc having an inner hole of a diameter designed to fit around a container in which oligonucleotide synthesis is performed. For example, the magnetic disc may be designed to fit around a synthesis column included in the kit. In another embodiment, the kit comprises at least one magnetic sleeve comprising a plurality of holes of a diameter designed to fit around a plurality of containers in which oligonucleotide synthesis can be performed. For example, the kit may include a magnetic sleeve designed to fit around wells of a multi-well plate (e.g., 96 well plate, 384 well plate, or 1,536 well plate), microcentrifuge tubes, or test tubes. In another embodiment, the kit comprises a device for reversibly demagnetizing and remagnetizing a magnet, as described herein.

These and other embodiments of the subject invention will readily occur to those of skill in the art in view of the disclosure herein.

### BRIEF DESCRIPTION OF THE FIGURES

FIG. 1A shows an illustration of an AB3900-style synthesis column with a commercial synthesis column with a NdFe8 sphere (3 mm diameter) in it serving as a solid substrate, which has been used for proof-of-concept studies. The chemicals used throughout the DNA synthesis protocol flow through this column as illustrated with the arrow. FIG. 1B shows a photo of an AB3900-style synthesis column with an NdFe8 sphere as a solid substrate. FIG. 1C shows the same column with the NdFe8 sphere from above. FIG. 1D shows a 40× magnification of the sphere coated with MyOne superparamagnetic beads before synthesis. FIG. 1E shows a 40× magnification of the sphere coated post-synthesis (in this case spheres were exposed to 20 cycles).

FIG. 2 shows an HPLC chromatogram of a T12mer synthesized on MyOne beads using NdFe8 spheres as a solid substrate. Note: altogether, six samples were generated, all of which, showed similar results.

FIG. 3 illustrates the benefits of direct coupling a C9 molecule to a hydroxylated surface followed by oligonucleotide synthesis, compared to the traditional approach of pre-synthesizing oligonucleotides with amino modifications and then coupling them to a carboxylated surface. The half circle in dark gray illustrates the surface of the bead and what chemical groups are present there. A major advantage of working with phosphoramidite chemical coupling compared to carbodiimide variants lies in the potential coupling efficiency. Coupling of the C9 (a smaller non-charged molecule) directly to the hydroxylated surface of the beads enables an efficient attachment. The oligonucleotides are then synthetized from the C9 support. In the traditional carbodiimide approach a synthetic amino-modified oligonucleotide is synthesized separately and then attached. This molecule is larger than the C9 molecule in size and also carries a highly negative charge (charge depending on size). Furthermore the surface charge of the carboxylated MyOne beads can be somewhat negative (increasing with increased pH) which will naturally repel any oligonucleotides in close proximity. The way to get around this is to perform the reactions at lower pH with certain potential difficulties depending on bead source and coupling efficiencies. This approach also somewhat limits the potential production-line automation advantages that are gained with the described direct synthesis approach.

FIG. 4 shows a device and method for separating beads from the substrate post-synthesis. A basic wiring for a demagnetizing/magnetizing apparatus is shown. When the circuit is switched to position 1 (demagnetize), an alternating current (AC) disrupts the magnetic domains of the substrate inside the titer plate passing through the coil. Switched to position 2, the electrons are forced into a direct current (DC) to re-magnetize the samples if applicable. Shown to the right is the housing where the titer plate passes horizontally through the magnetic field (depicted is the backside of the titer plate).

FIGS. 5A-5F show the high-throughput synthesis scheme. FIG. 5A shows an AB3900-style synthesis column modified to house three magnetic discs surrounding an inner column (˜10 mm diameter) as a proof-of-concept. FIG. 5B shows a column from the top with paramagnetic beads attached to the inner well wall by the outer magnets. FIG. 5C shows the retention of oxidizing reagent (reddish-brown) over beads. FIG. 5D shows that after 43 cycles (43 bases added in sequence), the beads are still bound along the well wall. Note: The AB3900 uses a top pressure drain system. FIG. 5E shows a 96-well permanent magnetic sleeve for placing around the PCR plate and 8-strip tube well bottoms. FIG. 5F shows a side view of the 96-well skirted PCR plate with a magnetic sleeve positioned half way down the wells.

### DETAILED DESCRIPTION

The practice of the present invention will employ, unless otherwise indicated, conventional methods of chemistry, biochemistry, molecular biology, and recombinant DNA techniques, within the skill of the art. Such techniques are explained fully in the literature. See, e.g., Protocols for Oligonucleotides and Analogs: Synthesis and Properties (Methods in Molecular Biology, S. Agrawal ed., Humana Press, 1993); Oligonucleotide Synthesis: Methods and Applications (Methods in Molecular Biology, P. Herdewijn ed., Humana Press, 2004); A. L. Lehninger, Biochemistry (Worth Publishers, Inc., current addition); Sambrook, et al., Molecular Cloning: A Laboratory Manual (3^{rd} Edition, 2001); Methods In Enzymology (S. Colowick and N. Kaplan eds., Academic Press, Inc.).

All publications, patents and patent applications cited herein, whether supra or infra, are hereby incorporated by reference in their entireties.

### I. DEFINITIONS

In describing the present invention, the following terms will be employed, and are intended to be defined as indicated below.

It must be noted that, as used in this specification and the appended claims, the singular forms “a,” “an” and “the” include plural referents unless the content clearly dictates otherwise. Thus, for example, reference to “a bead” includes a mixture of two or more beads, and the like.

The term “about,” particularly in reference to a given quantity, is meant to encompass deviations of plus or minus five percent.

The terms “polynucleotide,” “oligonucleotide,” “nucleic acid” and “nucleic acid molecule” are used herein to include a polymeric form of nucleotides of any length, either ribonucleotides or deoxyribonucleotides.

By “isolated” is meant, when referring to a polypeptide, that the indicated molecule is separate and discrete from the whole organism with which the molecule is found in nature or is present in the substantial absence of other biological macro-molecules of the same type. The term “isolated” with respect to a polynucleotide or oligonucleotide is a nucleic acid molecule devoid, in whole or part, of sequences normally associated with it in nature; or a sequence, as it exists in nature, but having heterologous sequences in association therewith; or a molecule disassociated from the chromosome.

“Purified polynucleotide” refers to a polynucleotide of interest or fragment thereof which is essentially free, e.g., contains less than about 50%, preferably less than about 70%, and more preferably less than about at least 90%, of the protein with which the polynucleotide is naturally associated. Techniques for purifying polynucleotides of interest are well-known in the art and include, for example, disruption of the cell containing the polynucleotide with a chaotropic agent and separation of the polynucleotide(s) and proteins by ion-exchange chromatography, affinity chromatography and sedimentation according to density.

The term “automated” is intended to include those methods by which an instrument is utilized to combine reagents in a stepwise fashion, thereby eliminating, in whole or in part, the need for an individual to mix or add reagents to each other. The term is further intended to include those purification steps and washing steps, which are often required during the synthesis of oligonucleotides, and steps, such as demagnetizing a magnet or removal of a magnet to allow collection of product, as described herein. The instrument is capable of performing these steps, as well as others, by programming, for example. For example, commercially available automated nucleic acid synthesizers (e.g., Applied Biosystems synthesizers AB394 and AB3900) or automated laboratory workstations (e.g., Beckman Coulter Biomek FX) can be adapted to prepare the oligonucleotides of interest with modifications to or addition of software instructing the instrument what reagents to add in a specified order and providing for control of a magnet.

The term “linker,” as used herein, refers to a molecule which reacts with a solid support that can be used to covalently link a compound (e.g., nucleotide, nucleoside phosphoramidite, or oligonucleotide) to a solid support. The linker should be able to react with the solid support and retain the ability to react with another reactive molecule. It will be appreciated by the skilled artisan that a variety of linkers can be used to covalently tether an oligonucleotide to a solid support. Linkers can be selected according to criteria such as length, chemical stability, or lability (where it is desired to cleave the compound from the solid support), and the like. Linker groups particularly useful for immobilizing oligonucleotides on a solid support include phosphoramidite linkers, such as 3-(4,4′-Dimethoxytrityloxy)propyl-1-[(2-cyanoethyl)-(N,N-diisopropyl)]-phosphoramidite (C3), 9-O-dimethoxytrityl-triethylene glycol, 1-[(2-cyanoethyl)-(N,N-diisopropyl)]-phosphoramiditepropanediol (C9), and 18-O-dimethoxytritylhexaethyleneglycol, 1-[(2-cyanoethyl)-(N,N-diisopropyl)]-phosphoramidite (C18).

As used herein, the term “probe” or “oligonucleotide probe” refers to a polynucleotide that contains a nucleic acid sequence complementary to a target nucleic acid analyte. Probes may be labeled in order to detect the target sequence. Such a label may be present at the 5′ end, at the 3′ end, at both the 5′ and 3′ ends, and/or internally.

The terms “hybridize” and “hybridization” refer to the formation of complexes between nucleotide sequences which are sufficiently complementary to form complexes via Watson-Crick base pairing. The terms are intended to refer to the formation of a specific hybrid between a probe and a target region.

The “melting temperature” or “Tₘ” of double-stranded DNA is defined as the temperature at which half of the helical structure of DNA is lost due to heating or other dissociation of the hydrogen bonding between base pairs, for example, by acid or alkali treatment, or the like. The Tₘ of a DNA molecule depends on its length and on its base composition. DNA molecules rich in GC base pairs have a higher Tₘ than those having an abundance of AT base pairs. Separated complementary strands of DNA spontaneously reassociate or anneal to form duplex DNA when the temperature is lowered below the Tₘ. The highest rate of nucleic acid hybridization occurs approximately 25 degrees C. below the Tₘ. The Tₘ may be estimated using the following relationship: Tₘ=69.3+0.41(GC) % (Marmur et al. (1962) J. Mol. Biol. 5:109-118).

As used herein, the terms “label” and “detectable label” refer to a molecule capable of detection, including, but not limited to, radioactive isotopes, fluorescers, chemiluminescers, chromophores, enzymes, enzyme substrates, enzyme cofactors, enzyme inhibitors, semiconductor nanoparticles, dyes, metal ions, metal sols, ligands (e.g., biotin, streptavidin or haptens) and the like. The term “fluorescer” refers to a substance or a portion thereof which is capable of exhibiting fluorescence in the detectable range. Particular examples of labels which may be used in the practice of the invention include, but are not limited to, horseradish peroxidase (HRP), SYBR® green, SYBR® gold, fluorescein, carboxyfluorescein (FAM), Alexa Fluor dyes, Cy3, Cy5, Cy7, 7-amino-4-methylcoumarin-3-acetic acid (AMCA), 5-(and-6)-carboxy-X-rhodamine, lissamine rhodamine, fluorescein-5-isothiocyanate (FITC), 7-diethylaminocoumarin-3-carboxylic acid, tetramethylrhodamine-5-(and-6)-isothiocyanate, 5-(and-6)-carboxytetramethylrhodamine, 7-hydroxycoumarin-3-carboxylic acid, 6-[fluorescein 5-(and-6)-carboxamido]hexanoic acid, N-(4,4-difluoro-5,7-dimethyl-4-bora-3a,4a diaza-3-indacenepropionic acid, eosin-5-isothiocyanate; erythrosine-5-isothiocyanate, 5-(and-6)-carboxyrhodamine 6G, CASCADE blue aectylazide, CAL Fluor Orange 560, CAL Fluor Red 610, Quasar Blue 670, tetramethyl rhodamine (TAMRA), 2′,4′,5′,7′-tetrachloro-4-7-dichlorofluorescein (TET), rhodamine, dansyl, umbelliferone, dimethyl acridinium ester (DMAE), Texas red, Pacific Blue, Pacific Orange, quantum dots, luminol, NADPH, and α-β-galactosidase.

### II. MODES OF CARRYING OUT THE INVENTION

Before describing the present invention in detail, it is to be understood that this invention is not limited to particular formulations or process parameters as such may, of course, vary. It is also to be understood that the terminology used herein is for the purpose of describing particular embodiments of the invention only, and is not intended to be limiting.

Although a number of methods and materials similar or equivalent to those described herein can be used in the practice of the present invention, the preferred materials and methods are described herein.

The present invention relates to a novel method for attaching oligonucleotides to a paramagnetic solid support, such as a paramagnetic bead or other paramagnetic particle. Conventional methods of attachment require that oligonucleotides be pre-synthesized with specific end modifications, which is laborious and expensive. Instead, the current inventors attached oligonucleotides to paramagnetic beads by direct synthesis of the oligonucleotides on the surface of the beads (see Example 1). An external magnet, capable of attracting and binding the beads, was used to hold the paramagnetic beads in place during solid-phase synthesis. A magnetic force was applied directly to the beads to prevent their loss, in particular, during reagent purge-to-waste steps that involved high-pressure drain or vacuum. This method can be adapted for use in any laboratory working with conventional synthesis automation, and can be employed, for example, with single columns and multi-well titer plates.

In order to further an understanding of the invention, a more detailed discussion is provided below regarding methods of synthesizing oligonucleotides on paramagnetic supports.

A. Oligonucleotide Synthesis Directly on a Paramagnetic Support

In one aspect, the invention includes a method for synthesizing one or more oligonucleotides of interest. The method comprises: a) providing a paramagnetic solid support comprising a substrate coating the surface of the solid support, wherein the substrate comprises one or more functional groups capable of covalent attachment to a linker; b) providing a magnet that attracts the paramagnetic solid support, whereby the paramagnetic solid support is immobilized on the surface of the magnet or a surface in contact with the magnetic field of the magnet; c) covalently attaching one or more phosphoramidite linkers to functional groups on the substrate to produce a derivatized support; d) reacting the derivatized support with a nucleoside phosphoramidite corresponding to the first nucleotide of the desired oligonucleotide sequence such that the nucleoside phosphoramidite attaches covalently to the linker; and e) adding nucleoside phosphoramidites stepwise to the growing nucleotide chain until an oligonucleotide having the desired sequence is produced. The oligonucleotide can subsequently be cleaved from the paramagnetic solid support or left as is, conjugated to the paramagnetic solid support for use in various applications. For example, oligonucleotides produced by this method can be used as capture oligonucleotides for isolation of nucleic acids of interest, primers for nucleic acid amplification or sequencing, and probes for detection of nucleic acids.

In the practice of the invention, nucleoside phosphoramidites are used because naturally occurring nucleotides are insufficiently reactive for efficient synthesis of oligonucleotides. Oligonucleotide synthesis may be performed in either the 5′ to 3′ direction or the 3′ to 5′ direction by suitable choice of nucleoside phosphoramidite reagents (e.g., 3′-[(2-cyanoethyl)-(N,N-diisopropyl)]-phosphoramidites for synthesis in the 3′ to 5′ direction or 5′-[(2-cyanoethyl)-(N,N-diisopropyl)]-phosphoramidites (Glen Research) for synthesis in the 5′ to 3′ direction). Depending on the direction of synthesis, the first nucleoside is attached to the paramagnetic solid support by a covalent ester linkage at either its 3′-hydroxyl or 5′-hydroxyl group. Nucleoside phosphoramidites corresponding to the desired sequence are added stepwise until the entire oligonucleotide has been assembled. Protecting groups may be attached to reactive functional groups present in the nucleosides to prevent unwanted side reactions during oligonucleotide synthesis. Such protecting groups can be removed after the desired oligonucleotide has been assembled. For a description of reagents, general reaction conditions, and equipment that can be used for oligonucleotide synthesis see e.g., Protocols for Oligonucleotides and Analogs: Synthesis and Properties (Methods in Molecular Biology, S. Agrawal ed., Humana Press, 1993); Oligonucleotide Synthesis: Methods and Applications (Methods in Molecular Biology, P. Herdewijn ed., Humana Press, 2004); Beaucage and Caruthers (1981) Tetrahedron Letters 22:1859-1862; Nielsen et al. (1986) Rec. Tray. Chim. Pays-Bas 105 (1):33-34; Nielsen et al. (1986) Nucl. Acids Res. 14 (18):7391-7403; Padmapriya et al. (1994) Antisense Res. Dev. 4:185-199; Ravikumar et al. (1994) Tetrahedron 50:9255-9266; Theisen et al. (1994) Nucleosides & Nucleotides 12:43; Iyer et al. (1995) Nucleosides & Nucleotides 14:1349-1357; Kuijpers et al. (1990) Nucl. Acids Res. 18:5197-5205; Ilyer et al. (1999) Curr. Opin. Molec. Therap. 1:344-358; Verma et al. (1998) Annu. Rev. Biochem. 67:99-134; Montseria et al. (1994) Tetrahedron 50:2617; Beaucage et al. (1993) Tetrahedron 49:1925-1963; Beaucage et al. (1993) Tetrahedron 49:6123-6194; Beaucage et al. (1992) Tetrahedron 48: 2223-2311; Englisch et al. (1991) Angew. Chemie Intl. Ed. Engl pp. 613-629; and Goodchild (1990) Bioconjugate Chemistry 1:165-187; herein incorporated by reference.

Non-nucleoside phosphoramidites may be used in the synthesis to produce modified oligonucleotides, for example, to add desired functional groups or detectable labels to the oligonucleotide. For example, non-nucleoside phosphoramidites can be used for attachment of a 5′-terminal phosphate, NH₂, SH, carbonyl, and carboxylic acid groups, carbon-carbon triple bonds, non-radioactive labels and quenchers (e.g., 6-FAM amidite, dabcyl amidite, biotin amidite), and hydrophilic and hydrophobic modifications (e.g., hexaethyleneglycol amidite and cholesterol amidite). See, e.g., Guzaev et al. (1995) Tetrahedron 51 (34):9375-9384; Sinha and Cook (1988) Nucl. Acids Res. 16 (6):2659-2669; Jones (1995) U.S. Pat. No. 5,391,785; Podyminogin (2001) Nucl. Acids Res. 29 (24):5090-5098; Lebedev (2007) Bioconjugate Chem. 18 (5):1530-1536; Alvira and Eritja (2007) Chemistry & Biodiversity 4 (12):2798-2809; U.S. Pat. No. 5,583,236; U.S. Pat. No. 6,114,518; U.S. Pat. No. 4,914,210; Durand et al. (1990) Nucl. Acids Res 18 (21): 6353-6359; International Application WO 2006078798; and Pon (1991) Tetrahedron Lett. 32 (14):1715-1718; herein incorporated by reference. The phosphoramidite approach can also be used to synthesize oligonucleotides having a variety of modified internucleoside linkages, such as oligonucleotide methylphosphonates, oligonucleotide phosphorothioates, and oligonucleotide N-alkylphosphoramidates. See e.g., Agrawal and Goodchild (1987) Tetrahedron Lett. 28: 3539-3542; Connolly et al. (1984) Biochemistry 23: 3443-3453; Jager el al. (1988) Biochemistry 27:7237-7246; herein incorporated by reference.

The paramagnetic solid support may comprise, for example, paramagnetic beads or non-spherical paramagnetic particles of any shape, including paramagnetic microparticles or nanoparticles (e.g., Dynabeads (Life Technologies), TurboBeads (TurboBeads Lk), SPHERO magnetic particles (Spherotech), and LuXSpheres (CardioGenics)). In certain embodiments, the paramagnetic solid support comprises a superparamagnetic material, which is magnetically displaceable but not permanently magnetizable. For example, the solid support may comprise a polymer containing superparamagnetic crystals, such as a maghemite or magnetite crystals (see, e.g., U.S. Pat. Nos. 4,654,267 and 8,227,262; herein incorporated by reference). The paramagnetic solid support may further comprise an inert polymeric coating on the surface of the support, for example, Teflon, perfluoroalkoxy, fluorinated ethylene propylene copolymer or polystyrene to protect the paramagnetic solid support from corrosion during oligonucleotide synthesis and prevent exposure of incipient oligonucleotides from exposure to metal ions from the interior of the paramagnetic solid support.

In one embodiment, the substrate functional group used for attachment of the linker is a hydroxyl group. Any suitable hydroxylated substrate (e.g., hydroxylated polystyrene) may be used for attachment of the linker. In certain embodiments, the phosphoramidite linker is between 30 and 60 atoms in length. Exemplary phosphoramidite linkers that can be used in the practice of the invention include 3-(4,4′-dimethoxytrityloxy)propyl-1-[(2-cyanoethyl)-(N,N-diisopropyl)]-phosphoramidite (C3), 9-O-dimethoxytrityl-triethylene glycol, 1-[(2-cyanoethyl)-(N,N-diisopropyl)]-phosphoramiditepropanediol (C9), and 18-O-dimethoxytritylhexaethyleneglycol, 1-[(2-cyanoethyl)-(N,N-diisopropyl)]-phosphoramidite (C18).

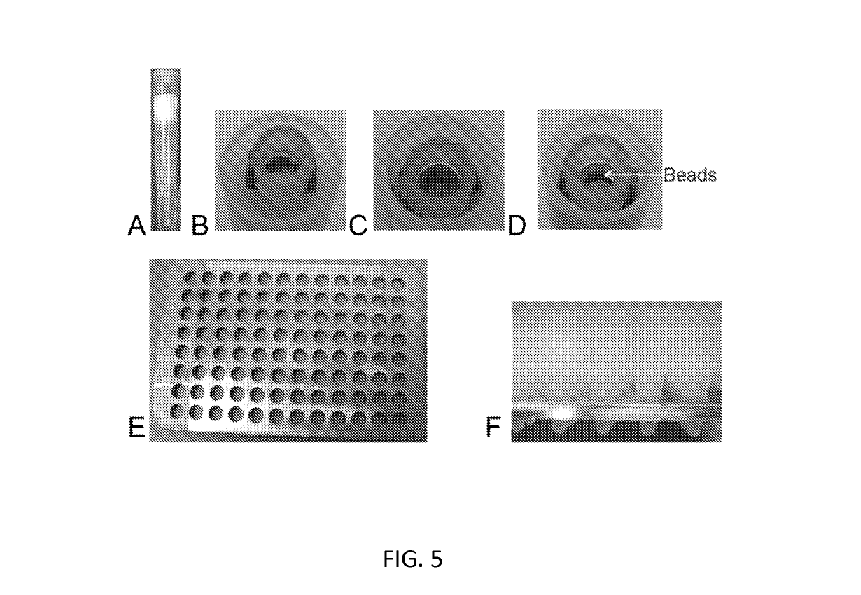

The magnet may be added to a container (e.g., column, microcentrifuge tube, test tube, or titer plate well) where oligonucleotide synthesis is carried out to immobilize the paramagnetic support. In certain embodiments, the magnet is a magnetic sphere or magnetic disc 1 mm to 6 mm in diameter. Alternatively, the magnet may surround a container or be placed in proximity to a container, such that the paramagnetic solid support is immobilized on a surface of the container. FIGS. 5A-5F illustrate some exemplary implementations, such as the use of magnetic discs surrounding a column, outer magnets attached to a column, and a magnetic sleeve comprising a plurality of holes designed to fit around titer plate wells or tubes of a certain diameter.

In certain embodiments, the method further comprises temporarily demagnetizing the magnet to release the paramagnetic solid support from the magnet. For example, the magnet can be demagnetized by switching the magnetic poles of the magnet by moving the magnet back and forth near an external magnet or exposing the magnet to alternating current. In one embodiment, the magnet is passed through a conducting coil carrying an alternating current such that the magnet becomes demagnetized. Alternatively, the magnet can be demagnetized by heating the magnet to a temperature at or above the Curie point of the magnet. If the paramagnetic solid support is immobilized during oligonucleotide synthesis by indirect contact with the magnet, that is, by immobilization on a surface in contact with the magnetic field of the magnet (e.g., surface of a column, tube, or titer plate well surrounded by the magnet or next to or in contact with the magnet), the paramagnetic solid support can be released from the surface simply by removing the magnet.

In another embodiment, oligonucleotide synthesis, as described herein, is automated by use of a commercial nucleic acid synthesizer or automated laboratory workstation. For example, commercially available automated nucleic acid synthesizers (e.g., Applied Biosystems AB394 and AB3900) or automated laboratory workstations (e.g., Beckman Coulter Biomek FX) can be adapted to prepare oligonucleotides of interest with modifications to or addition of software instructing the instrument what reagents to add in a specified order and providing for control of a magnet. Such an instrument can be used to combine reagents in a stepwise fashion, thereby eliminating, in whole or in part, the need for an individual to mix or add reagents to each other. Purification steps and washing steps, which are required during the synthesis of oligonucleotides, can also be performed by such an instrument. The automated system may use columns, tubes, high-density multi-well plates (e.g., 96, 384 and 1536-well), or any other suitable container for oligonucleotide synthesis.

In particular, the automated system can be adapted to control a magnet. In one embodiment, the automated system temporarily demagnetizes a magnet to allow collection of the paramagnetic support (e.g., paramagnetic beads, microparticles, or nanoparticles). Demagnetization can be accomplished by passing a column or multi-well plate through a coil with an alternating current so that the magnet is demagnetized, thereby releasing the paramagnetic support into solution. Alternatively, a removable magnet (e.g., outer magnet, magnetic disc, or magnetic sleeve) designed to fit around or attach to the outside of wells, tubes, columns, or other container (see, e.g., FIGS. 5B, 5C and 5D) can be used. In this case, the paramagnetic support binds to the surface of a container in contact with the magnet or magnetic field of the magnet, and remains there throughout repeated synthesis cycles. The paramagnetic support can be released into solution simply by removing the magnet.

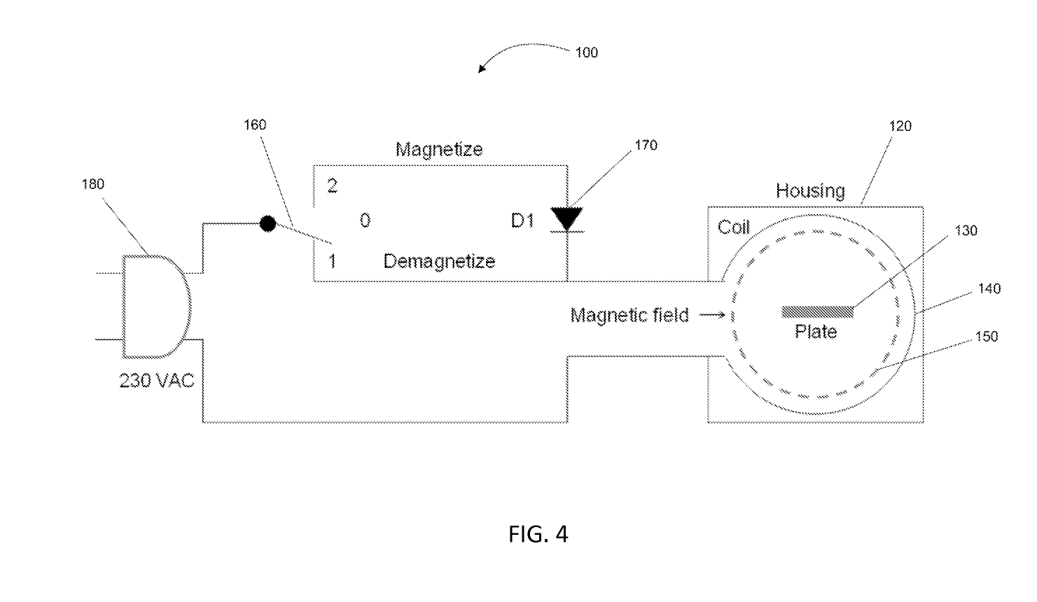

An exemplary device for reversibly demagnetizing and remagnetizing a magnet for controlling release of a paramagnetic solid support from a magnet is described in Example 1. The device 100 comprises: a) a housing 120; b) a conducting coil 140 positioned inside the housing 120, wherein the conducting coil 140 is capable of generating a magnetic field 150, whereby a magnet positioned within the magnetic field is demagnetized when alternating current (AC) flows through the conducting coil and remagnetized when direct current (DC) flows through the conducting coil; c) an AC power supply 180; d) a rectifier 170 that converts AC to direct current (DC); and e) a circuit connected to the AC power supply and the conducting coil and having a switch 160 that controls whether or not AC from the AC power supply passes through the rectifier before flowing through the conducting coil, wherein the position of the switch determines whether AC or DC is supplied to the conducting coil (see FIG. 4). The device may further comprise a platform positioned such that the platform is in contact with the magnetic field generated by current flowing through the conducting coil, wherein the platform can be used for placement of a reaction container for performing synthesis on a paramagnetic solid support, as described herein. In certain embodiments, the device is incorporated into an automated system, such as a commercial nucleic acid synthesizer or automated laboratory workstation.

B. Kits

Materials and reagents for performing oligonucleotide synthesis, as described herein, can be provided in kits with suitable instructions. The materials and reagents for oligonucleotide synthesis (e.g., nucleoside phosphoramidites, phosphoramidite linkers, paramagnetic beads, and magnets) may be contained in separate containers. Additionally, instructions (e.g., written, tape, VCR, CD-ROM, DVD, Blu-ray, etc.) for oligonucleotide synthesis may be included in the kit. The kit may also contain other packaged reagents and materials (e.g., multi-well plates, synthesis columns, solvents, protecting groups, acids, bases, and other reagents for performing oligonucleotide synthesis, and the like).

The kit may include magnets in various forms, such as but not limited to magnetic spheres, magnetic discs, magnetic brackets, and magnetic sleeves. In one embodiment, the kit comprises at least one magnetic disc having an inner hole of a diameter designed to fit around a container in which oligonucleotide synthesis is performed. For example, the magnetic disc may be designed to fit around a synthesis column included in the kit. In another embodiment, the kit comprises at least one magnetic sleeve comprising a plurality of holes of a diameter designed to fit around a plurality of containers in which oligonucleotide synthesis can be performed. For example, the kit may include a magnetic sleeve designed to fit around wells of a multi-well plate (e.g., 96 well plate, 384 well plate, or 1,536 well plate), microcentrifuge tubes, or test tubes.

In another embodiment, the kit comprises a device for reversibly demagnetizing and remagnetizing a magnet, as described herein.

C. Applications

Direct in situ oligonucleotide synthesis on paramagnetic beads (or other paramagnetic particles) allows for immediate use of the newly synthesized oligonucleotides in various applications. For example, oligonucleotide synthesis may be used to create custom primers for nucleic acid amplification (e.g., PCR) or sequencing (e.g., pyrosequencing or ion torrent sequencing). Primers can be specifically designed to allow particular segments of DNA to be amplified or sequenced. In addition, oligonucleotides can be designed to incorporate other useful sequences, such as restriction sites, tags, or reporter genes into a nucleic acid sequence during amplification.

Custom oligonucleotides can also be designed as probes that bind to a region of DNA that is complementary to the oligonucleotide sequence. Such probes can be used to isolate DNA of a specific sequence. For this purpose, the use of oligonucleotides conjugated to a paramagnetic solid support is particularly useful. The conjugated oligonucleotide can be incubated with a DNA-containing sample in a buffer, under conditions suitable for hybridization. The captured DNA, bound to the conjugated oligonucleotide, can then be collected using a magnet that attracts the paramagnetic support.

Hybridization conditions can be adjusted to detect a range of nucleic acid variants with homology to an oligonucleotide probe. Moderately stringent hybridization conditions allow detection of a target nucleic acid sequence of at least 15 nucleotides in length having at least approximately 70% sequence identity with the sequence of the selected nucleic acid probe. Stringent hybridization conditions allow detection of target nucleic acid sequences of at least 15 nucleotides in length having a sequence identity of greater than 90% with the sequence of the selected nucleic acid probe. Hybridization conditions useful for probe/target hybridization, where the probe and target have a specific degree of sequence identity, can be determined as is known in the art (see, for example, Nucleic Acid Hybridization: A Practical Approach, editors B. D. Hames and S. J. Higgins, (1985) Oxford; Washington, D.C.; IRL Press). Hybrid molecules can be formed, for example, on a solid support, in solution, and in tissue sections. The formation of hybrids can be monitored by inclusion of a reporter molecule, typically, in the probe. Such reporter molecules or detectable labels include, but are not limited to, radioactive elements, fluorescent markers, and molecules to which an enzyme-conjugated ligand can bind.

With respect to stringency conditions for hybridization, it is well known in the art that numerous equivalent conditions can be employed to establish a particular stringency by varying, for example, the following factors: the length and nature of the probe and target sequences, base composition of the various sequences, concentrations of salts and other hybridization solution components, the presence or absence of blocking agents in the hybridization solutions (e.g., formamide, dextran sulfate, and polyethylene glycol), hybridization reaction temperature, and time parameters, as well as, varying wash conditions. The selection of a particular set of hybridization conditions is well known (see, for example, Sambrook, et al., Molecular Cloning: A Laboratory Manual, 3rd Edition, 2001).

In addition, oligonucleotides bound to a paramagnetic solid support can be used to isolate DNA-binding proteins. An oligonucleotide, conjugated to a paramagnetic solid support, can be incubated with a sample containing a DNA-binding protein in a buffer under conditions appropriate for the protein under investigation. The bound protein is collected with a magnet utilizing the paramagnetic properties of the solid support. The captured protein can then be eluted from the oligonucleotide (e.g., by adjusting salt concentration or pH) for downstream applications and detection.

The methods of the invention can readily be adapted to multiplex assays and high-throughput screening. Large numbers of oligonucleotides can be attached to paramagnetic beads (or other paramagnetic support) for highly parallel analysis of multiple nucleic acids simultaneously. In particular, bead-based arrays comprising conjugated oligonucleotides can be assembled for massively parallel high throughput screening. The methods of the invention are especially useful for multiplexed PCR amplification, sequencing, nucleic acid hybridization assays, and single nucleotide polymorphism genotyping.

### III. EXPERIMENTAL

Below are examples of specific embodiments for carrying out the present invention. The examples are offered for illustrative purposes only, and are not intended to limit the scope of the present invention in any way.

Efforts have been made to ensure accuracy with respect to numbers used (e.g., amounts, temperatures, etc.), but some experimental error and deviation should, of course, be allowed for.

### Example 1

### Direct Oligonucleotide Synthesis on Paramagnetic Beads Using Filterless Solid-Phase Phosphoramidite Chemistry

Here we describe a novel way of attaching oligonucleotide ligands to paramagnetic beads. Micro- and nanometer-sized beads are used extensively for a vast variety of molecular diagnostic assays, ranging from suspension bead arrays to Next Generation Sequencing sample preparation materials. One of the key activities involved with preparing the beads for downstream applications involves conjugating a ligand (e.g., nucleic acid or proteins) to the surface of the bead. This has previously been accomplished with a cumbersome and inefficient method of chemical conjugation via carbodiimide mediated amide bond formation. Furthermore, in the case of oligonucleotide ligands, they need to be pre-synthesized, which involves multi-step procedures. Our novel solution for attaching oligonucleotides to paramagnetic beads is instead accomplished by direct synthesis of oligonucleotides on the surface of the beads. By applying a magnetic force directly to the beads we prevent their loss caused by high-pressure drain or vacuum during reagent purge-to-waste steps in the synthesis cycle. This is achieved by using an external magnet (e.g., sphere or disc 1-6 mm in diameter) to which the beads are attracted and bound; the magnet in this case acts as the solid substrate. What makes this a powerful research tool is its versatility and availability. We envision using this method in any laboratory working with conventional synthesis automation, as employed for single columns (e.g., AB394, and AB3900) and for multi-well titer plates (e.g., 96, 384, and 1,536 well formats).

Material and Methods

Synthesis on MyOne Beads

Hydroxyl coated MyOne beads (Life Technologies, Carlsbad, Calif.) were washed several times with deionized water to remove BSA (used to prevent clumping during storage). Single magnetic spheres (K&J Magnetics) (0.125″ diameter, Neodymium-NdFeB) were then placed inside empty synthesis columns (Biosearch Technologies, Novato, Calif.) on top of a 40 μm filter. One μl of the original MyOne stock solution (10⁷ beads/μl) was used to coat the surface of the solid substrate (permanent magnetic sphere). Prior to synthesis, magnetic sphere-bound MyOne beads were then washed several times with acetonitrile to remove excess water and residual BSA. Using the manufacturer's recommended protocol (modified for increased coupling times) all samples were first tethered with a non-cleavable triethylene glycol linker (Spacer Phosphoramidite 9) (10-1909-02, Glen Research, Sterling, Va.) to the hydroxylated surface of the MyOne beads. For control samples, a base-labile succinate anhydride linker (ChemGenes, Wilmington, Mass.) was added before the first base of each sequence for cleavage during ammonolysis. All sequences were then synthesized using 5′ to 3′ phosphoramidites (Glen Research, Sterling, Va.) using a default 50 nmole-scale protocol.

Post-Synthesis

Following synthesis, control samples (T12mers) with magnetic spheres were placed inside 1.5 ml Eppendorf tubes to which 80 μl ammonium hydroxide (NH₄OH) was added to cleave the oligonucleotides from the MyOne beads; the tops were sealed with tape to prevent ammonia evaporation during incubation at room temperature (RT) for 10 minutes. Samples were then centrifuged for 5 seconds to collect the free oligonucleotides. This was repeated twice. After which point, the synthesis columns were removed, and the eppendorf tubes placed inside a thermomixer for further incubation at 70° C. for 30 minutes to remove the cyanoethyl protecting groups.

Method for Separating Beads from the Substrate Post-Synthesis

For our experiments, we rapidly switched the poles of the spheres using an external magnet temporarily demagnetizing them long enough to aspirate the MyOne beads in solution. The beads were then released in a 1.5 mL eppendorf tube containing 160 μl of 1× Wash/Storage Buffer [PBS, supplemented with 0.2% Tween-20 (pH 7.7)]. Three rounds of washing were performed with 200-μl 1× Wash/Storage Buffer before using a standard DYNAL Invitrogen bead separator magnetic rack (Life Technologies, Carlsbad, Calif.). The final bead pellet was then resolved in 20-μl× Wash/Storage Buffer.

Oligonucleotide synthesis verification using reverse-phase High Pressure Liquid Chromatography (HPLC)

Control T12 samples with a succinate bridge were run on HPLC to confirm their identity and purity with standards using a DNASep C-18 column at 60° C. with UV detection at 260λ; running buffers consisted of water, acetonitrile, triethyl ammonium acetate and EDTA (HPLC unit and operating software used for sample analysis consisted of the Transgenomic Wave System).

Oligonucleotide Synthesis Verification Using Fluorescent Quantification

A 41 bp primer sequence, Primer A′ TTTTTTTTTTTTTTTGTCGGAGACACGCAGGGATGAGATGG (SEQ ID NO:1) was synthesized directly on the hydroxylated MyOne beads, as described above. Post synthesis beads were washed three times with 1× Wash/Storage Buffer and remaining beads were resuspended in a 3-μl aliquot of 1× Wash/Storage Buffer (density unknown). As a comparator Amino-Primer A′ (/5AmMC6/TTTTTTTTTTTTTTTGTCGGAGACACGCAGGGATGAGATGG) was synthesized with a 5′ amino modifier C6 (IDT technologies, Coralville, Iowa) and covalently attached to MyOne Carboxylic Acid beads (Life Technologies, Carlsbad, Calif.) via a traditional 2-step carbodiimide chemical protocol (Nakajima and Ikada (1995) Bioconjug. Chem. 6:123-130; Gilles et al. (1990) Anal. Biochem. 184:244-248; Sehgal and Vijay (1994) Anal. Biochem. 218:87-91; Szajáni et al. (1991) Appl. Biochem. Biotechnol. 30:225-231) using EDC (Sigma-Aldrich, St. Louis, Mo.), 25 mM MES, pH 6 (Sigma-Aldrich, St. Louis, Mo.), and washed with 50 mM Tris pH 7.5 (Sigma-Aldrich, St. Louis, Mo.). The oligonucleotide containing beads were then quantified with the Qubit 2.0 Fluorometer (Life Technologies, Carlsbad, Calif.) together with the Qubit ssDNA HS assay kit (Life Technologies, Carlsbad, Calif.). For control samples, 3-μl of non-synthesized hydroxylated MyOne beads (approximately 10 M beads/μl) and 3-μl non-attached carboxylated MyOne bead (approximately 10 M beads/μl) were used as negative controls and also quantified in the Qubit 2.0 Fluorometer with the Qubit ssDNA HS assay kit. Each measured component was repeated in triplicate and each Qubit 2.0 measurement was performed three subsequent times.

Primer A CCATCTCATCCCTGCGTGTCTCCGAC (SEQ ID NO:2, IDT technologies, Coralville, Iowa) was synthesized as a template positive control with a complementary sequence to the region downstream of the 15 polyT region on Primer A′ sequence. Post synthesis beads were washed three times with 1× Annealing Buffer [PBS, supplemented with 0.2% Tween-20 (pH 7.7)] and the remaining beads were suspended in a 3-μl aliquot (density unknown). Primer A [100 μM] was added to the aliquots for a total volume of 6-μl Annealing of Primer A to Primer A′ was performed in the GeneAmp PCR system 9700 (Life Technologies, Carlsbad, Calif.) at 95° C. for 2 minutes followed by 37° C. for 2 minutes and cooling to room temperature. The beads were subsequently washed three times with 1× Wash/Storage Buffer before quantification with the Qubit dsDNA HS assay kit (Life Technologies, Carlsbad, Calif.) on the Qubit 2.0 Fluorometer. As a negative control sample 3-μl of non-synthesized hydroxylated MyOne beads (approximately 10 M beads/μl) were used and quantified in the Qubit 2.0 Fluorometer. Each measured component was repeated in triplicate and each Qubit 2.0 measurement was performed in three subsequent times.

Results and Discussion

The Filterless Solid-Phase Oligonucleotide Synthesis Platform

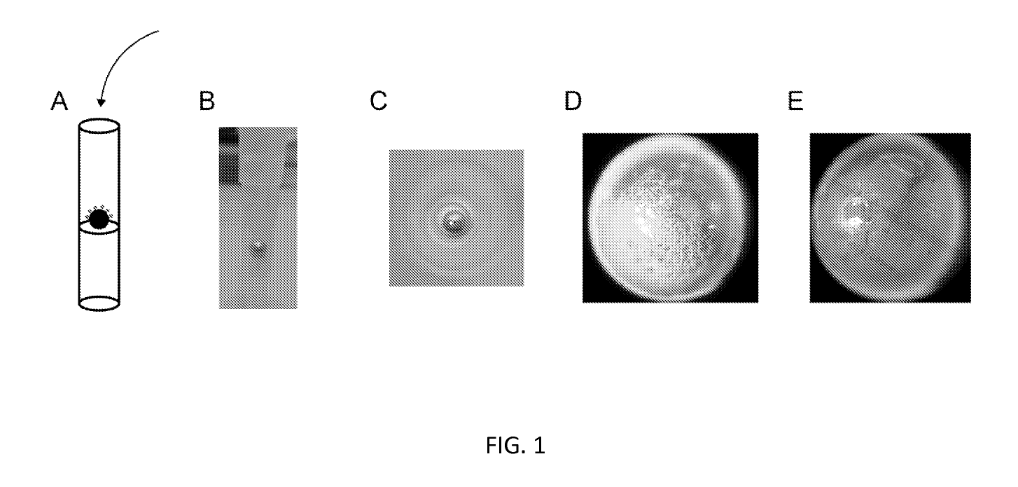

We chose the magnetic approach to oligonucleotide synthesis because containment of submicron paramagnetic beads is not possible using a filtered well or column (as in traditional DNA synthesis (Hughes et al. (2011) Chapter 12—Gene Synthesis: Methods and Applications 1st ed. Elsevier Inc.)) due to flow restrictions. It would require a filter porosity of ≦2 μm to retain the beads; however, organic solvents used in oligonucleotide synthesis (acetonitrile, trichloroacetic acid, pyridine, etc.) will not pass through the filter even under elevated pressure. Furthermore, submicron beads have the potential to clog the filter pores increasing resistance to reagent flow. Therefore, using an external magnetic source (NdFeB sphere 0.125″ diameter) placed inside a synthesis column with a porous filter to retain the sphere (FIGS. 1A-1C) was the preferred alternative.

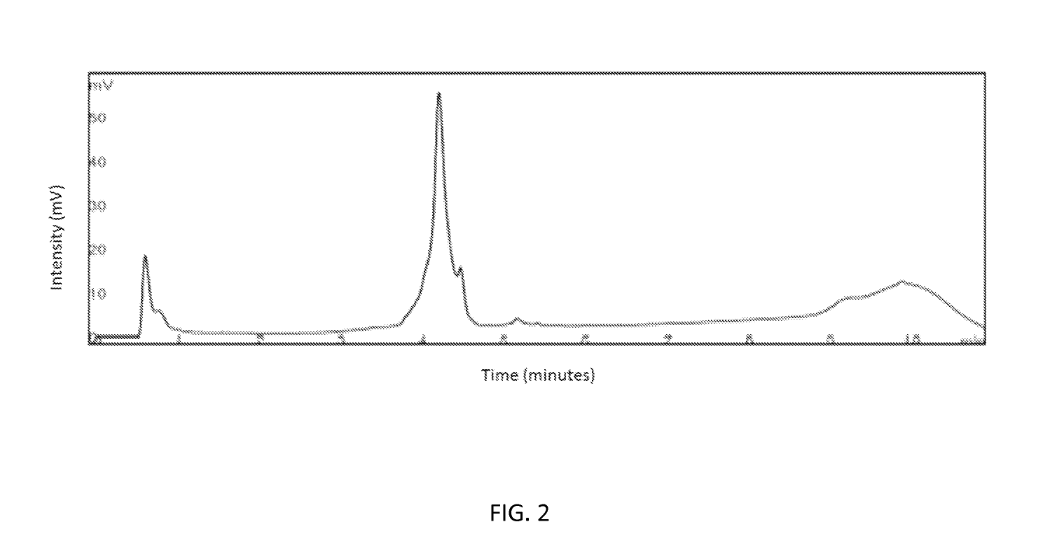

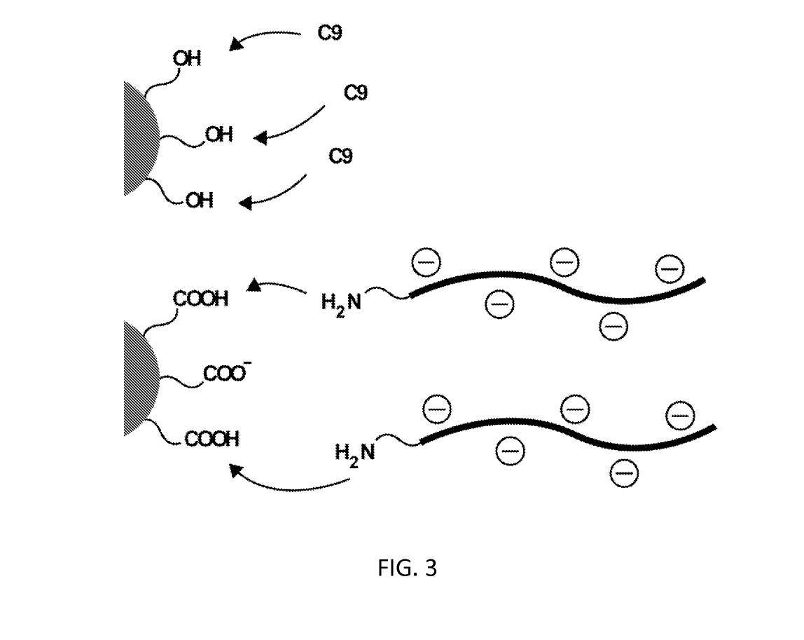

In our research, we used superparamagnetic beads (MyOne), which are coated with hydroxylated polystyrene. The hydroxyl groups are an ideal starting point for phosphoramidite coupling to the surface. Because it is required that the generated oligonucleotides remain tethered to the beads, we chose a chemical linker that was non-cleavable during ammonolysis (used for base deprotection). This was also important to avoid steric hindrance associated with neighbouring base-base interaction; coupling nucleosides too near the surface results in poor yields. Based on commercially available phosphoramidite linkers, our choices included 3-(4,4′-Dimethoxytrityloxy)propyl-1-[(2-cyanoethyl)-(N,N-diisopropyl)]-phosphoramidite (C3), 9-O-Dimethoxytrityl-triethylene glycol, 1-[(2-cyanoethyl)-(N,N-diisopropyl)]-phosphoramiditepropanediol (C9), and 18-O-Dimethoxytritylhexaethyleneglycol, 1-[(2-cyanoethyl)-(N,N-diisopropyl)]-phosphoramidite (C18). Previous literature studies of these linkers indicated that a spacer between 30 and 60 atoms with a low negative charge (FIG. 3) maintained its hydrophilic properties; therefore, C9 was found optimal for coupling to an aminated polypropylene support). Though the hydroxyl density is unknown for the MyOne beads, making it difficult to calculate product yield, we discovered that coupling with the C9 linker was effective (FIG. 2). To demonstrate this, we introduced a base-labile succinate linker (5′-DMT thymidine succinyl hexamide phosphoramidite), which allowed for oligonucleotide release during ammonolysis for HPLC analysis.

Furthermore, all synthesis was done in the 5′ to 3′ direction (5′-3′ phosphoramidites, Glen Research, VA). In order for the polymerase extension to occur in a sequencing reaction, for example, where target DNA/RNA is hybridized to the immobilized oligonucleotide, a 3′ recess must be available. Extended spacing from the surface also plays a critical role in enzyme/DNA/RNA binding efficiency due to minimized steric hindrance. Such efficiencies would likely increase if a polyT (10-15 bases) were coupled to the C9 linker as well.

While there is flexibility to synthesize in several different column and plate configurations, we chose to focus on the column-based AB3900 used in routine synthesis (50 nm-scale) as our target instrument for generating oligonucleotides on paramagnetic beads. To date we have shown that superparamagnetic beads (MyOne) will readily bind to a permanent magnetic sphere, which acts as the synthesis solid substrate. To test the magnetic stability through repeated wash and drain steps, we subjected test samples to a 40-cycle synthesis run. Here we only loaded bare hydroxylated beads onto the spheres and with an initial loading of approximately 1(10)⁷ beads, we were left with approximately 1(10)⁵ beads at the end of the run (FIGS. 1D and 1E). Generating T12mers, however, final yields were much higher (˜5(10)⁶). The reason for product loss in both cases is most likely explained by a decreased magnetism of the outer layers of beads. Because the surface of the sphere is flat, beads stack on top of one another during the loading process (FIG. 2B); as such, some beads are stripped from the surface of the sphere during high-pressure drain steps of each synthesis cycle. Consequently, full-length product (FLP) could be lost yielding more n−1 strands as the layers are stripped away. To circumvent this, we are collaborating with 4^{th} State Inc. (Belmont, Calif.), which specializes in Plasma-Enhanced Chemical Vapor Deposition (PE-CVD) to coat the magnetic spheres, with a chemically inert polymeric resin (i.e. non-porous Teflon, perfluoroalkoxy, fluorinated ethylene propylene copolymer or polystyrene). The aim is (i) to protect the DNA from metal ions produced during corrosion due to prolonged acid exposure, and (ii) to create a mesh that will increase the surface area for the beads to attach and minimize their loss during synthesis.

A major advantage of working with phosphoramidite chemistry compared to carbodiimide variants lies in the potential coupling efficiency (FIG. 3). In the phosphoramidite variant, we coupled the C9 (a smaller non-charged molecule) directly with the hydroxylated surface of the beads, which enabled an efficient attachment. The oligonucleotides were then synthetized from the C9 support. In the traditional carbodiimide approach, a synthetic amino-modified oligonucleotide is synthesized separately and then attached. This molecule is larger than the C9 molecule in size and also carries a highly negative charge (charge depending on size). Furthermore the surface charge of the carboxylated MyOne beads can be somewhat negative (increasing with increased pH), which will naturally repel any oligonucleotides in close proximity. The way to get around this is to perform the reactions at lower pH, with certain potential difficulties depending on bead source and coupling efficiencies. This approach also somewhat limits the potential production-line automation advantages that are gained with the described direct synthesis approach.

Device and Method for Separating Beads from the Substrate Post-Synthesis

In order to retrieve the oligonucleotide-bound beads post-synthesis, magnetic properties of the substrate must first be disrupted. This can be accomplished by either of two methods: (i) Heat or (ii) demagnetization by an alternating current (AC). If a permanent magnet is heated at or beyond its Curie point or is exposed to AC, it will temporarily or permanently lose all magnetic properties (mceproducts.com) due to forced randomization of their magnetic domains. Though we experimented with heat application, our results were inconclusive. For our work, beads were released from the spheres by rapidly switching their poles. This was done by moving the sphere inside the Eppendorf tube (with 160 μl H₂O) back and forth over an external magnet. This temporarily disrupted the magnetic domains of the sphere allowing for release of the beads into solution long enough to rapidly aspirate them by pipette.

In addition to processing single column synthesis on paramagnetic beads, we are adapting this technology to high-density titer plates (96, 384 and 1536-well) for maximum throughput. For the latter case, an automated system to temporarily demagnetize the spheres and aspirate or collect paramagnetic beads may be required. Our first proposition is to build an apparatus that operates on an alternating/direct current (FIG. 4); here, the 1536-well titer plate, for example, is passed through a coil with an alternating current so the spheres are demagnetized, thus releasing the beads into solution. For the 96 and 384-well platforms, however, we envision using a permanent magnetic bracket (FIGS. 5 E and 5F) that would fit around the outside of the wells. As shown in FIGS. 5B, 5C and 5D, paramagnetic beads readily bind to the well surface where they remain throughout repeated synthesis cycles. Releasing the beads into solution in this embodiment is accomplished by removing the magnetic bracket. Furthermore, to retrieve the samples while the substrate is demagnetized, the beads can be easily aspirated into a buffer using a manual or automated pipettor. Fluidic workstations such as the Biomek FX are outfitted for highly paralleled liquid delivery, mixing and transfer that accommodate nearly every plate style and well configuration.

How efficient the AC process is at demagnetizing the substrate is largely unexplored. Therefore, we will (i) choose a substrate with the ideal strength magnetism (too strong and the beads will not release even when passed through an alternating current), (ii) establish the optimum magnetic field strength by adding more turns of wire to the coil, using a larger wire diameter, or change the geometry of the coil, and (iii) setup the apparatus so that the plate position within the magnetic field can be adjusted, as well as its speed and distance passing through the coil.

Oligonucleotide Synthesis Verification Using Fluorescent Quantification

To verify the existence of synthesized oligonucleotide Primer A′ on the hydroxylated MyOne beads, a fluorescent dye was used that effectively binds to single-stranded DNA molecules. Post-synthesis beads were extensively washed to remove any non-attached nucleic acid molecules. As a comparator, native non-synthesized beads were used. A fluorescent quantification assay was then performed using similar amounts of beads to see if any dye had attached to the synthesized oligonucleotides, and thus measure DNA concentration present. When comparing the native Hydroxylated MyOne beads to the post-synthesis ones, a clear difference in DNA concentration was observed: 9.71 ng/ml versus 29.8 ng/ml respectively (Table 1). As a reference carboxylated MyOne beads were coupled to a pre-synthesized amino-modified Primer A′ oligonucleotide molecule. When comparing non-coupled carboxylated MyOne beads to the oligonucleotide coupled ones, a clear difference in DNA concentration was observed: 6.99 ng/ml versus 20.3 ng/ml (Table 1). The DNA concentration was approximately 30% higher for the synthesized beads compared the coupled ones, which might account for an improved efficiency for the described production method.

To verify accurate sequence content on the synthesized bead, a non-modified Primer A oligonucleotide (complementary to Primer A′) was individually hybridized in over-saturated concentrations to the beads. Following the annealing procedure, the beads were washed extensively to remove all traces of Primer A. The beads were subsequently quantified with a dye to detect the presence of double-stranded DNA, which would only be present if Primer A had annealed to the Primer A′ target strand on the beads (Table 1). When compared with native non-synthesized beads (both with and without Primer A annealing), the post-synthesis beads showed the strongest DNA concentration with 4.07 ng/ml versus 0.95 ng/ml (native beads no annealing) and 0.82 ng/ml (native beads with annealing). This provides proof that the proper sequence had been synthesized on the beads, as it was able to retain annealing of Primer A to create double-stranded DNA.

Oligonucleotide Synthesis Verification Using DNA Sequencing

Ultimately we would want to verify the exact sequence content on each synthesized bead. The best means for this would be to actually sequence each individual bead. In a setup similar to the Primer A hybridization experiment, we would synthesis a longer oligonucleotide fragment to both contain a priming site (Primer A) and a downstream sequence to be read by an instrument. We have evaluated the possibility of doing so in a classic pyrosequencing (Ronaghi (2001) Genome Research 11:3-11; Gharizadeh et al. (2006) J. Biotechnology 124:504-511) instrument in an attempt to read all beads in a pool. However, the nature of the beads quenches the light signal obtained in the Pyrosequencing reaction and is not a preferred measurement source. A better approach would be to try and read the sequence in the Life Technologies Ion Torrent Personal Genome Machine (PGM) (Rothberg et al. (2012) Nature 475:348-352; Merriman et al. (2012) Electrophoresis 33:3397-3417). The instrument accepts smaller beads (˜2 μm) and reads the signal via an electronic pH measurement scheme, rather than a light based reaction. The only issue is the compatibility with our bead source, as the current generation of PGM instruments only accepts acrylamide beads. We are currently investigating possible upcoming sequencing instruments to assess compatibility with our beads.

### CONCLUSION

While use of paramagnetic beads in sample preparation and detection (Pyrosequencing, MagArray, etc.) has many advantages, conventional methods of bead attachment require that oligonucleotides be pre-synthesized with specific end modifications; as such, this is laborious and expensive, demanding many hours of preparation. Therefore to mainstream the process, we have introduced a method of direct oligonucleotide synthesis onto paramagnetic beads (i.e. MyOne). Because bead diameter is often within a few microns or less, filter-based solid-phase DNA synthesis is not possible because of reagent flow restrictions. However, by applying an external magnetic sphere to which the beads are bound, synthesis can occur with minimal loss of product during wash and drain steps of each cycle. We have demonstrated proof-of-concept by fluorescent quantification and HPLC analysis of samples that were both permanently tethered to the beads and those that were released through ammonolysis via a succinate linkage. In both cases, a C9 (triethylene glycol) spacer was introduced at the surface to which the first base was coupled using 5′-3′ phosphoramidites. Furthermore, we anticipate a greater demand for future applications where small volume, multi-well titer plates (96, 384 and 1536 platforms) will greatly increase throughput for large-scale projects. As such, we have proposed development of a device and method for rapidly releasing samples from their magnetic spheres by applying either heat or an alternating current to disrupt their magnetic domains long enough to collect the beads for further downstream processing.

While the preferred embodiments of the invention have been illustrated and described, it will be appreciated that various changes can be made therein without departing from the spirit and scope of the invention.

TABLE 1
A summary of the results from the DNA quantification experiments
of pre- and post-synthesis beads.

| | BEAD TYPE | SOURCE | PROCESS | DYE | DNA CONC. |
|---|---|---|---|---|---|
| I. | Carboxylated | Native beads | Wash | ssDNA | 6.99 ng/ml |
| II. | Carboxylated | Coupled ligand | Wash | ssDNA | 20.3 ng/ml |
| III. | Hydroxylated | Native beads | Wash | ssDNA | 9.71 ng/ml |
| IV. | Hydroxylated | Post-synthesis | Wash | ssDNA | 29.8 ng/ml |
| V. | Hydroxylated | Native beads | Wash | dsDNA | 0.95 ng/ml |
| VI. | Hydroxylated | Native beads | Anneal | dsDNA | 0.82 ng/ml |
| | | | Primer A | | |
| VII. | Hydroxylated | Post-synthesis | Anneal | dsDNA | 4.07 ng/ml |
| | | | Primer A | | |

(I) Native carboxylated MyOne beads that were washed, but never went through any ligand coupling reactions. This is a negative control to check for background signal, and quantification was based on a single-strand DNA dye.
(II) Carboxylated MyOne beads to which the amino modified synthetic oligo Primer A′ was chemically coupled. After coupling, the beads were washed, and quantification was based on a single-strand DNA dye.
(III) Native hydroxylated MyOne beads that were washed, but never went through any oligonucleotide synthesis procedure. This is a negative control to check for background signal, and quantification was based on a single-strand DNA dye.
(IV) Hydroxylated MyOne beads post-synthesis of sequence Primer A′. The beads were washed after the synthesis has been performed, and quantification was based on a single-strand DNA dye.
(V) Native hydroxylated MyOne beads that were washed, but never went through any oligonucleotide synthesis procedure. This is a negative control to check for background signal, and quantification was based on a double-strand DNA dye.
(VI) Native hydroxylated MyOne beads that were washed after annealing Primer A oligonucleotide to the beads. These beads never went through any oligonucleotide synthesis procedure. This is also a negative control to check for background signal, and quantification was based on a double-strand DNA dye.
(VII) Hydroxylated MyOne beads post-synthesis of sequence Primer A′. The beads were washed after the synthesis had been performed, and after annealing Primer A oligonucleotide. Quantification was based on a single-strand DNA dye.

What is claimed is:

1. A method for synthesis of an oligonucleotide, the method comprising:
a) providing a paramagnetic solid support comprising a substrate coating the surface of the solid support, wherein the substrate comprises one or more functional groups capable of covalent attachment to a linker;
b) providing a magnet that attracts the paramagnetic solid support, whereby the paramagnetic solid support is immobilized on the surface of the magnet or a surface in contact with the magnetic field of the magnet;
c) covalently attaching phosphoramidite linkers to functional groups on the substrate to produce a derivatized support;
d) reacting the derivatized support with a nucleoside phosphoramidite corresponding to the first nucleotide of the desired oligonucleotide sequence such that the nucleoside phosphoramidite attaches covalently to a phosphoramidite linker; and
e) adding nucleoside phosphoramidites stepwise to the growing nucleotide chain until an oligonucleotide having the desired sequence is produced.

2. The method of claim 1, wherein the functional group used for attachment of the linker is a hydroxyl group or an amino group.

3. The method of claim 1, wherein the substrate is a hydroxylated substrate.

4. The method of claim 3, wherein the substrate comprises hydroxylated polystyrene.

5. The method of claim 1, wherein the phosphoramidite linker is between 30 and 60 atoms in length.

6. The method of claim 1, wherein the phosphoramidite linker is selected from the group consisting of 3-(4,4′-Dimethoxytrityloxy)propyl-1-[(2-cyanoethyl)-(N,N-diisopropyl)]-phosphoramidite (C3), 9-0 Dimethoxytrityl-triethylene glycol, 1-[(2-cyanoethyl)-(N,N-diisopropyl)]-phosphoramiditepropanediol (C9), and 18-O-Dimethoxytritylhexaethyleneglycol, 1-[(2-cyanoethyl)-(N,N-diisopropyl)]-phosphoramidite (C18).

7. The method of claim 1, wherein the magnet is a magnetic sphere or magnetic disc 1-6 mm in diameter.

8. The method of claim 1, wherein the magnet is a magnetic disc having an inner hole of a diameter designed to fit around a container in which oligonucleotide synthesis is performed.

9. The method of claim 8, wherein the magnetic disc is designed to fit around a column in which oligonucleotide synthesis is performed.

10. The method of claim 1, wherein the magnet is a magnetic sleeve comprising a plurality of holes of a diameter designed to fit around a plurality of containers in which oligonucleotide synthesis is performed.

11. The method of claim 10, wherein the magnetic sleeve is designed to fit around wells of a multi-well plate.

12. The method of claim 11, wherein the multi-well plate is a 96 well plate, 384 well plate, or 1,536 well plate.

13. The method of claim 10, wherein the magnetic sleeve is designed to fit around micro centrifuge tubes or test tubes.

14. The method of claim 1, further comprising temporarily demagnetizing the magnet to release the solid support from the magnet.

15. The method of claim 14, wherein demagnetizing the magnet comprises switching the magnetic poles of the magnet by moving the magnet back and forth near an external magnet.

16. The method of claim 14, wherein demagnetizing the magnet comprises heating the magnet to a temperature at or above the Curie point of the magnet.

17. The method of claim 14, wherein demagnetizing the magnet comprises exposing the magnet to an alternating current.

18. The method of claim 17, wherein the magnet is passed through a conducting coil carrying an alternating current such that the magnet is demagnetized.

19. The method of claim 1, further comprising removing the magnet to release the paramagnetic solid support from a surface that was in contact with the magnet.

20. The method of claim 19, wherein the magnet is a magnetic disc, magnetic sleeve, or outer magnet.

21. The method of claim 1, further comprising cleaving the oligonucleotide from the support.

22. The method of claim 1, wherein the magnet is contained within a synthesis column, microcentrifuge tube, test tube, or titer plate well.

23. The method of claim 1, wherein oligonucleotide synthesis is performed in the 5′ to 3′ direction.

24. The method of claim 1, wherein oligonucleotide synthesis is performed in the 3′ to 5′ direction.

25. The method of claim 1, wherein the support is a superparamagnetic bead.

26. The method of claim 1, wherein the support comprises an inert polymeric coating on the surface of the support.

27. The method of claim 26, wherein the polymeric coating comprises Teflon, perfluoroalkoxy, fluorinated ethylene propylene copolymer or polystyrene.

28. The method of claim 1, further comprising adding one or more non-nucleoside phosphoramidites.

29. An automated system capable of synthesizing one or more oligonucleotides according to the method of claim 1 comprising a conducting coil carrying an alternating current capable of demagnetizing a magnet.

30. An automated system capable of synthesizing one or more oligonucleotides according to the method of claim 1 comprising one or more removable magnets capable of attaching to one or more containers in which oligonucleotide synthesis is performed.

31. The automated system of claim 30 comprising at least one magnetic disc comprising an inner hole of a diameter designed to fit around a container in which oligonucleotide synthesis is performed.

32. The automated system of claim 31, wherein the magnetic disc is designed to fit around a column in which oligonucleotide synthesis is performed.

33. The automated system of claim 30 comprising at least one magnetic sleeve comprising a plurality of holes of a diameter designed to fit around a plurality of containers in which oligonucleotide synthesis is performed.

34. The automated system of claim 33, wherein the magnetic sleeve is designed to fit around wells of a multi-well plate.

35. The automated system of claim 34, wherein the multi-well plate is a 96 well plate, 384 well plate, or 1,536 well plate.

36. The automated system of claim 33, wherein the magnetic sleeve is designed to fit around microcentrifuge tubes or test tubes.

37. A kit comprising one or more magnets and reagents for performing oligonucleotide synthesis according to the method of claim 1.

38. The kit of claim 37 comprising superparamagnetic beads.

39. The kit of claim 37, wherein at least one magnet is a magnetic sphere or magnetic disc 1-6 mm in diameter.

40. The kit of claim 37, wherein at least one magnet is a magnetic disc having an inner hole of a diameter designed to fit around a container in which oligonucleotide synthesis is performed.

41. The kit of claim 40, wherein the magnetic disc is designed to fit around a column in which oligonucleotide synthesis is performed.

42. The kit of claim 37, wherein at least one magnet is a magnetic sleeve comprising a plurality of holes of a diameter designed to fit around a plurality of containers in which oligonucleotide synthesis is performed.

43. The kit of claim 42, comprising a magnetic sleeve designed to fit around wells of a multi-well plate.

44. The kit of claim 43, wherein the multi-well plate is a 96 well plate, 384 well plate, or 1,536 well plate.

45. The kit of claim 42, wherein the magnetic sleeve is designed to fit around micro centrifuge tubes or test tubes.

46. A device for reversibly demagnetizing and remagnetizing a magnet for controlling release of a paramagnetic solid support from the magnet or attachment of a paramagnetic solid support to the magnet, the device comprising:
a) a housing;
b) a conducting coil positioned inside the housing, wherein said conducting coil is capable of generating a magnetic field, whereby the magnet, when positioned within the magnetic field, is demagnetized when alternating current (AC) flows through the conducting coil and remagnetized when direct current (DC) flows through the conducting coil;
c) an AC power supply;
d) a rectifier that converts AC to direct current (DC); and
e) a circuit connected to the AC power supply and the conducting coil and having a switch that controls whether or not AC from the AC power supply passes through the rectifier before flowing through the conducting coil, wherein the position of the switch determines whether AC or DC is supplied to the conducting coil.

47. The device of claim 46, further comprising a platform positioned within the magnetic field generated by current flowing through the conducting coil.

48. An automated system capable of synthesizing oligonucleotides comprising the device of claim 46.
